(19) United States
(12) Patent Application Publication
(10) Pub. No.: US 2011/0165037 A1
(43) Pub. Date: Jul. 7, 2011

(54) INTERFACES THAT ELIMINATE NON-SPECIFIC ADSORPTION, AND INTRODUCE SPECIFIC INTERACTIONS

(76) Inventors: Rustem F. Ismagilov, Chicago, IL (US); Jason Eugene Kreutz, Chicago, IL (US)

(21) Appl. No.: 12/986,777

(22) Filed: Jan. 7, 2011

Related U.S. Application Data
(60) Provisional application No. 61/335,570, filed on Jan. 7, 2010.

(51) Int. Cl. B01L 3/00; C07C 271/20; C07C 41/09

(52) U.S. Cl. 422/502; 560/159; 568/677

(57) ABSTRACT

A microfluidic system comprising a microchannel, a carrier fluid comprising a fluorinated oil in the microchannel, at least one plug comprising an aqueous plug-fluid in the microchannel and substantially surrounded on all sides by the carrier-fluid, and a fluorinated surfactant comprising a functional group capable of selectively binding a target molecule is disclosed. A compound for use therewith and a method of synthesizing a fluorinated surfactant are also provided.

### RELATED APPLICATIONS

This application claims priority to U.S. Provisional Patent Application Ser. No. 61/335,570, incorporated by reference herein in its entirety.

### FEDERALLY SPONSORED RESEARCH OR DEVELOPMENT

This invention was made with government support under grant numbers GM074961, EB000557 and DP1OD003584 awarded by the National Institutes of Health (NIH). The government has certain rights in the invention.

### TECHNICAL FIELD

The present disclosure relates to microfluidic systems, and in particular, to interfaces that eliminate non-specific adsorption and introduce specific interactions.

### BACKGROUND

Interfaces are critical to consider when using biological solutions. Biofouling, nonspecific adsorption and denaturing of proteins, peptides, cells and other biological species can interfere with “normal” interactions. This can lead to observations, results, or conclusions that are not relevant to in vivo situations where these interfaces are absent. Developing a system that minimizes the types of interfaces exposed to the system, and that is able to control the remaining interfaces is desirable.

### BRIEF SUMMARY

In one aspect, a microfluidic system includes a microchannel, a carrier fluid comprising a fluorinated oil in the microchannel, at least one plug comprising an aqueous plug-fluid in the microchannel and substantially surrounded on all sides by the carrier-fluid, and a fluorinated surfactant comprising a functional group capable of selectively binding a target molecule.

In some embodiments, the microfluidic system includes a second fluorinated surfactant. The second fluorinated surfactant may have a functional group that does not significantly bind to the target molecule.

In some embodiments, the target molecule is a biological molecule.

In some embodiments, the target molecule is water soluble.

In some embodiments, the functional group of the fluorinated surfactant is selected from a nitrilotriacetate, an iminediacetate a triazacyclononane, a biotin derivative, a glutathione derivative, a maltose derivative, a thioredoxin tag, a FLAG tag, a hemaglutinin tag, and an OmpA signal sequence tag.

In another aspect, a compound of the formula

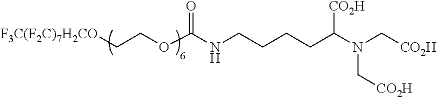

and salts thereof are disclosed.

In another aspect, a method of synthesizing a fluorinated surfactant of the formula CF₃(CF₂)ₙ(CH₂)ₘO(CH₂CH₂O)ₚH, where n is an integer from 1 to 20, m is an integer from 1 to 4, p is an integer from 3 to 6 is disclosed. The fluorinated surfactant is prepared by coupling a compound of the formula CF₃(CF₂)ₙ(CH₂)ₘOH and a compound of the formula HO(CH₂CH₂O)ₚH.

### DETAILED DESCRIPTION

Microfluidic systems that minimize the types of interfaces exposed in the system and which enable better control of those interfaces are disclosed. By surrounding plugs in a microfluidic system with, for example, fluorinated oil, all interfaces except the fluorous-aqueous interface are removed. Fluorinated oils are preferred, as they have very low solubility towards aqueous and organic components, leading to minimal contamination and loss of material. In certain embodiments, coating the fluorous-aqueous interface with specific surfactants provides control of the surface chemistry and can reduce nonspecific adsorption and/or introduce specific interface-target molecule interactions.

Nonspecific interactions can be reduced through the use of fluorous surfactants, amphiphiles, or detergents, which may be referred to as “Rf-inert”, which can comprise a fluorinated tail, which may be referred to as “Rf”, and a head group that assembles to form an inert surface at the fluorous-aqueous (F:A) interface that is resistant to adsorption of biological species including, for example, proteins and cells. The tail, Rf, can comprise, for example, CF₃(CF₂)ₙ(CH₂)ₘ—, where, for example, n is an integer between 0 and 20 and m is an integer between 1 and 4, or fluorinated ethers, such as Krytox fluorinated ethers, for example those comprising 14 to 44 monomeric units, or Fomblin fluorinated ethers. Head groups of Rf-inert can comprise, for example, a polyethylene glycol group, an oligoethylene group, a zwitterion, such as poly((3-(methacryloylamino)propyl)-dimethyl(3-sulfopropyl)ammonium hydroxide (poly(MPDSAH)) (as described in Cho, W. K.; Kong, B. Y.; Choi, I. S., Langmuir 2007, 23, 5678-5682), or materials, including sugars, zwitterions and sorbitol derivatives, described in U.S. Pat. No. 7,494,714 and U.S. Pat. No. 7,276,286 and U.S. Pat. No. 6,972,196 U.S. Ser. No. 11/124,022, incorporated by reference herein in their entirety, permethylated D-Dulcitol-Derived or D-Mannitol-Derived Polymer (MPDME), such as are described in Metzke, M.; Guan, Z., Biomacromolecules 2008, 9, 208-215, a mannitol group, a permethyl sorbitol group, tri(sarcosine) or N-acetylpiperazine, as described in Ostuni, E.; Chapman, R. G.; Liang, M. N.; Meluleni, G.; Pier, G.; Ingber, D. E.; Whitesides, G. M., Langmuir, 2001, 17, 6336-6343.

The present invention can be used with, for example, microfluidic technology, including plug technology, such as is disclosed in U.S. Pat. Nos. 7,129,091 and 7,655,470, and patent applications PCT/US08/71374, PCT/US08/71370, PCT/US07/26028, PCT/US09/46255, U.S. Ser. No. 12/777,099, U.S. 61/340,872, U.S. 61/335,570, U.S. 61/262,375, U.S. Ser. No. 12/670,739, U.S. Ser. No. 12/670,725, U.S. Ser. No. 12/520,027, U.S. Ser. No. 12/162,763, U.S. Ser. No. 11/174,298, U.S. Ser. No. 10/765,718, and U.S. Ser. No. 11/589,700 all incorporated by reference herein in their entireties, or SlipChip technology, as disclosed in PCT/US2010/028316, incorporated by reference herein in its entirety.

The present invention can be used in sensors to reduce or eliminate nonspecific or background adsorption or interactions. Other sensor technologies have used rinsing to eliminate or reduce background signal. The ability to reduce or eliminate this undesired adsorption allows for detection of weaker binding events and labile interactions that, for example, processes involving washing or rinsing can miss. Exemplary diagnostic systems to which the present invention can be applied are described in U.S. patent application Ser. Nos. 12/678,014, 11/698,802 and 11/698,757, incorporated by reference herein in their entireties. Rinsing can result in the loss of real interactions if those interactions are weak or very labile. The background signal can be minimized by using an inert interface, as is formed by the use of Rf-inert, and, optionally, a specific interaction can be introduced that allows for selective adsorption to the interface, increasing the analyte signal. Enhancement of the analyte signal can be sufficient to remove the need for rinsing, allowing the detection of important but weak or labile binding events that rinsing or nonspecific adsorption can mask.

In certain embodiments of the invention, the high surface area to volume ratio of plugs favorably enhances the distribution of molecules in a plug to increase sensitivity. In certain preferred embodiments, the solution is depleted of a target analyte and substantially all of the target analyte is located at the plug surface, leading to a strong signal. It will be apparent to one skilled in the art that the target analyte can be bound to a fluorophore, for example a fluorophore compatible with FRET, for detection. Other exemplary detection strategies include having a fluorescently labeled capture agent complexed with a quencher-labeled analyte, wherein when the analyte is present, quencher is displaced and the label on the capture agent is detectable. In certain embodiments, the long axis of a plug can be oriented perpendicular to the detection source, increasing the effective path length for enhanced detection. Manipulation of the plug geometry by manipulating the channel geometry can enhance this effect. In certain embodiments, by altering droplet geometry, for example, by sending a plug through a constriction, an analyte-complexing surfactant may be selectively concentrated in certain parts of the plug leading to concentration of the target molecule and enhancement of the analyte signal. An analyte-complexing surfactant can also be concentrated via plug flow. For example, during flow, surfactant and thus the target analyte can be concentrated at the back of the plug leading to enhanced signal.

Other applications include protein crystallization experiments in which it is desirable to increase, control or reduce interface induced nucleation. Other applications include: enzyme-based assays in which it is desirable to prevent the denaturing of proteins; cell-based assays in which exposure to a single biocompatible interface can improve cell viability and eliminate or reduce unwanted interactions and side effects; screening for interactions between amyloid proteins and potential drug species, since, by avoiding aggregation induced by interfacial adsorption one can scan systematically for molecules which alter and preferably inhibit the intrinsic self-aggregation process of amyloid peptides and proteins; studying interactions between amyloid proteins and other biological species; studying interactions between Aβ protein and other species by eliminating or reducing unnatural aggregation due to air water interfaces or other artificial interfaces. Another application is studying the interaction of Aβ with ganglioside clusters by generating precise clusters of gangliosides within droplets, as gangliosides have been shown to interact with Aβ, and clustering of gangliosides in cholesterol rich lipid rafts is thought to be biologically relevant. Identifying a critical cluster size can lead to insights in the disease progression. Such experiments can also generate specific Aβ species for, for example, studying their toxicity. Potential drugs for the inhibition of the interaction between Aβ and ganglioside clusters can also be screened using certain embodiments of the present invention. Other interactions between amyloid proteins can also be studied, for example potential interactions between Tau and Aβ.

Certain embodiments of the present invention can be used to detect signs of Alzheimer's disease in brain fluids. Proteomic and metabolic changes in the brain due to amyloid diseases can be monitored by certain embodiments of the present invention. For example, cerebrospinal fluid (CSF) or interstitial fluid (ISF) can be sampled, and multiple analytical assays, for example, ELISA assays, can be performed in parallel.

The present invention can be used in combination with a chemistrode, which is disclosed in U.S. Ser. No. 12/737,058, incorporated herein by reference in its entirety.

It will be apparent to one skilled in the art that any functional groups that are able to bind and/or capture a specific species in solution can be linked to a fluorinated tail such as Rf to generate molecules which may be termed “Rf-capture”. Such molecules can be used to concentrate and orient such species at the fluorinated phase/aqueous phase interface. Such fluorinated capturing components can be used in the absence or presence of inert fluorinated surfactants.

Certain embodiments of the present invention can be used for PCR, for example by using Rf-inert to reduce or eliminate deactivation of enzymes or adsorption of DNA or primers.

It will be apparent to one skilled in the art that a number of species can be used to link a capture moiety, such as a chelating group capable of binding to a His tag, to a fluorinated tail. Preferably, the capture species is kept sufficiently far from the fluorinated phase/aqueous phase interface that the interface does not interfere with binding (as might be the case with large proteins) and the binding species still maintains some degree of freedom. Possible linking groups for maintaining sufficient spacing include, but are not limited to, the following, alone or in combination: oligoethylene glycol groups, for example, oligoethylene glycol groups containing two to six glycol units; a carbamate group, for example one derived from carbonyl diimidazole; an acetate group, for example one derived from an α-bromoester; a succinate group, such as one derived from succinic anhydride; and other simple linkers such as an ether, an amine, an ester, an amide, an α-hydroxy amide, an ω-carboxyamide or an aromatic group.

It will be apparent to one skilled in the art that “capture” groups that can be linked to fluorinated tails can comprise, for example: divalent cation binding groups, such as nitrilotriacetate (NTA, for binding to His tags), iminediacetate (IDA), or triazacyclononane (TACN); specific peptide or protein binding tags, such as biotin (for binding to streptavidin or avidin derivatives), glutathione (for binding to proteins or other substances linked to glutathione-S-transferase), maltose (for binding to proteins or other substances linked to maltose binding protein), thioredoxin tag, FLAG tag, hemaglutinin tag, OmpA signal sequence tag and the like. Capture groups can comprise, for example, protein or nucleic acid species, including antibodies, antigens, aptamers, DNA, RNA, or other oligonucleotides or peptides. Capture groups can also comprise sugars, including, for example gangliosides (for example, GM1), maltose, glucose, sialic acid or galactose. It will be apparent to one skilled in the art that each such capture group will be chosen to capture a substance with an affinity for the chosen capture group. Other capture agents (tags) and their uses that can be used as part of the present invention are disclosed in U.S. application Ser. No. 12/415,988, incorporated by reference herein in its entirety. Capture agents can also be peptides, proteins, antibodies or aptamers. Conjugates of fluorinated groups and proteins are disclosed in U.S. patent application Ser. Nos. 12/201,894, 11/520,182, and U.S. Pat. No. 5,055,562, herein incorporated by reference in their entirety. Aptamers are disclosed in U.S. Pat. Nos. 7,700,759, 5,773,598, 5,496,938, 5,580,737, 5,654,151, 5,726,017, 5,786,462, 5,503,978, 6,028,186, 6,110,900, 6,124,449, 6,127,119, 6,140,490, 6,147,204, 6,168,778, and 6,171,795, 5,472,841, 5,567,588, 5,582,981, 5,637,459, 5,683,867, 5,705,337, 5,712,375, and 6,083,696 herein incorporated by reference in their entirety.

Certain embodiments of the present invention can be used as sensors. Selectively concentrating a substance at an interface with an appropriate capture element can increase a detectable signal. For example, a light-adsorbing interface can be aligned perpendicular to a photo detector (e.g., a detector comprising microscope optics) providing improved detection. The channel geometry that a plug passes through at the point of detection can be altered to increase the detectability of a signal. In certain embodiments, the mobility of the surfactants may allow for selective concentration of Rf-capture when plug geometry is altered, thus further increasing the detectability of a target molecule. For example, by flowing plugs, fluorinated surfactants comprising capture groups together with the bound target analyte can be concentrated at the back of the plug, allowing for an increase in the detectability of a signal. Such techniques may also use inert fluorinated surfactants to minimize the background noise, for example by reducing denaturation, or concentration of non-target species at the fluorinated phase/aqueous phase interface.

Certain embodiments of the invention can be used for protein crystallization. As described herein, surfactants comprising a capture agent can be used to selectively and specifically concentrate and orient a target analyte at the fluorinated phase/aqueous phase interface, leading to an increased rate or chance of nucleation and improved crystal quality.

Certain embodiments of the invention can be used to manipulate protein aggregation. For example, this technique can be used to explore interactions with specific surface chemistries. Gangliosides, for example, have been shown to interact with Aβ. Functionalizing the fluorinated phase/aqueous phase interface with gangliosides or other species can provide information on how they affect Aβ aggregation. In addition, certain embodiments of the invention can be used to investigate whether conditions such as dialysis related amyloidosis (DRA) are due to protein exposure to an unnatural surface, or if some critical component is getting concentrated or depleted by the procedure. The present invention provides the ability to cleanly control interfacial interactions to study such systems.

Certain embodiments of the invention can be used for screening for drug-peptide interactions. Drugs that inhibit protein-protein interactions can be screened. One can use such embodiments to screen for drugs that interfere with biologically relevant interactions that facilitate, for example, the aggregation of Aβ or other amyloid proteins.

Certain embodiments of the invention can be used for enzyme assays. Some enzymes and proteins do not always perform their function in solution and often are located or interact with other species at membranes or other biological interfaces. The localization, concentration and orientation that can result from this, as well as potential allosteric interactions, can greatly alter the activity of an enzyme. Thus studies that explore and account for these types of interactions are more relevant to actual events in vivo than experiments done in bulk solution (See Gureasko, J.; Galush, W. J.; Boykevisch, S.; Sondermann, H.; Bar-Sagi, D.; Groves, J. T.; Kuriyan, J. Nat. Struct. Mol. Biol. 2008, 15, 452-461). The present invention can be used to precisely control interactions at interfaces for this type of study.

Certain embodiments of the invention can be used for multi-component structure formation. The assembly of multicomponent structures at or in interfaces is critical for many systems including the immune response and cellular signaling. Formation of the immunological synapse is one example.

Certain embodiments of the invention can be used for cell-based or bacterial assays, including selective activation of cells that have certain binding affinities. All cells have functionalities that are important for recognition and cellular function. Some cells need to adhere to a surface to properly function. The adhesion in biological systems is not always on a flat or immobile surface as is often the case with other model systems, so cellular interactions with curved and/or mobile interfaces are of interest.

Certain embodiments of the invention can be used to extract or capture cells (for example, pathogens, circulating tumor cells, or immune cells) or other biological species (for example, proteins, toxins, or inflammatory cytokines) from biological samples such as blood or CSF, using capture agents such as antibodies, antigens, nanobodies, aptamers, and the like that are anchored with a fluorinated tail.

Certain embodiments of the invention can be used for nanoparticle or other material synthesis. Fluorinated surfactants comprising capture groups can serve as an organizing scaffold, nucleating, and/or stabilizing agent to control formation of specific species, polymorphs or surfaces.

Certain embodiments of the invention can be used for multiphase transport, reaction or separation. For example, surfactant functionalities can dictate the reactivity of plugs. If different surfactants are localized to different plugs, then multistep reactions can occur as reagents and/or intermediates are transported and/or transferred between plugs using previously described techniques.

Certain embodiments of the invention can be used to create a barrier using surfactant functionalities that permit certain species to freely transport between plugs while other species are trapped. Such control of permeability offers a system that mimics some components of cellular behavior.

In certain embodiments of the present invention, a microfluidic method is used to control interfacial chemistry in nanoliter droplets to enable miniaturized in vitro measurements of protein aggregation. Measuring aggregation of proteins experimentally is applicable to understanding the biophysics of, for example, amyloidosis, and for developing methods for detection and treatment of, for example, amyloid diseases. In typical in vitro aggregation experiments, adsorption of amyloid peptides to the air/water and other solid/water interfaces accelerates peptide aggregation by enhancing nucleation. Intrinsic stochasticity of nucleation phenomena implies that obtaining statistically significant data often requires multiple experiments performed in parallel, making miniaturization of these experiments attractive. The interfacial effects, however, become even more pronounced upon miniaturization of aggregation experiments, as miniaturization leads to an increase of the surface-to-volume ratio. Miniaturization of aggregation experiments is especially desirable for samples available only in small volumes, such as cerebrospinal fluid (CSF) from, for example, mice. CSF is known to contain components that inhibit formation of amyloid aggregates, and the balance of inhibitory and pro-aggregation activities changes with aging and progression of disease. Analysis of CSF is therefore useful for biomarker discovery and monitoring effects of drugs in animal models of Alzheimer's diseases. For mouse experiments, such panels of experiments are not easily done with standard multiwell-plate in vitro assays that require tens of microliters of sample, given that the volume of CSF obtainable from a single mouse is only a few microliters.

In order to miniaturize aggregation experiments while controlling the interfacial chemistry one can use a plug-based microfluidic approach in, for example, poly(dimethylsiloxane) (PDMS) microfluidic devices modified with Teflon tubing. Exemplary plugs can comprise nanoliter fluorocarbon-surrounded aqueous droplets formed in the flow of immiscible fluids inside microfluidic channels. Once an amyloid peptide, for example, Aβ₄₀, is encapsulated inside a plug, it is protected from the surfaces of microchannels by a layer of fluorocarbon, and the surface chemistry of the aqueous-fluorous interface, rather than the aqueous-channel interface, becomes relevant to the peptide. It was demonstrated that the adsorption of Aβ₄₀ to the aqueous-fluorous interface can be minimized by comparing the behavior of Aβ₄₀ labeled with HiLyte-488 at the N-terminus at two liquid/liquid interfaces: (i) an aqueous peptide/fluorocarbon interface without surfactant and (ii) an aqueous peptide/n-C₈F₁₇CH₂-OEG₃-protected fluorocarbon interface. n-C₈F₁₇CH₂-OEG₃ is an amphiphilic fluorinated surfactant, CF₃(CF₂)₇CH₂O(CH₂CH₂O)₃H, that can be added to the carrier fluid, and without wishing to be bound by theory, is thought to assemble spontaneously at the aqueous-fluorous interface, and present triethyleneglycol groups to the aqueous phase, thereby preventing or reducing protein adsorption.

In plugs containing no n-C₈F₁₇CH₂-OEG₃, a fluorescence signal of the labeled Aβ₄₀ peptide increased at the plug edges, indicating adsorption of the peptide at the interface 2 hours after encapsulation. By contrast, in plugs with n-C₈F₁₇CH₂-OEG₃, the fluorescence signal of the labeled peptide was evenly distributed, demonstrating that n-C₈F₁₇CH₂-OEG₃ prevents Aβ₄₀ adsorption to the interface. The effectiveness of n-C₈F₁₇CH₂-OEG₃ was a function of its concentration in the carrier fluid. To decrease exposure of Aβ₄₀ to the channel walls prior to plug formation, one can insert Teflon tubing into the peptide inlet. To confirm that Aβ₄₀ is not lost during the plug formation process, the fluorescence intensity of 5-40 μM fluorescein-labeled Aβ₄₀ was measured after encapsulation within the plugs containing n-C₈F₁₇CH₂-OEG₃. A linear increase in intensity that coincided with the increase of intensity of fluorescein (combined R²=0.99), a compound that does not adsorb to surfaces in our devices, was found.

Certain embodiments of the present invention can be used to monitor aggregation kinetics. For example, the differences in the aggregation kinetics of Aβ₄₀ in plugs with and without n-C₈F₁₇CH₂-OEG₃ were monitored using Thioflavin T (ThT), a widely used dye that has been used in other systems to monitor aggregation by observing the increase of ThT fluorescence upon binding to amyloid aggregates. The aggregation kinetics of Aβ₄₀ depend on nucleation and are typically described by a sigmoidal curve. As a control, 50 μM Aβ₄₀ aggregation kinetics in 100 μL volumes in a well plate were monitored. A lag period of ˜10 hours was observed before the ThT fluorescence indicated the start of Aβ₄₀ aggregation. In plugs with a fluorocarbon/water interface, the lag time of the aggregation reaction shortened to ˜2 hours, correlating with the adsorption of the peptide to the fluorocarbon/water interface and also with the increased surface-to-volume ratio in 10 nL volumes. In contrast, the lag time for aggregation of Aβ₄₀ in plugs with n-C₈F₁₇CH₂-OEG₃ present increased to many days. After 250 hours, only 3 plugs out of 15 showed evidence of Aβ₄₀ aggregation. Even after 2 months, only 8 plugs out of 15 showed evidence of Aβ₄₀ aggregation. Thus, control of the interfacial chemistry decreases the aggregation kinetics of Aβ₄₀ by at least an order of magnitude.

It has been shown that the system reliably handles aggregation experiments with small volumes using CSF from a single mouse (5 μL). The relative ability of CSF sampled from two mouse strains to inhibit Aβ₄₀ aggregation was also studied. In this comparison, nontransgenic (wt) mice and ceAPPswePS1ΔE9/TTR−/− transgenic mice that exhibit amyloid deposition throughout the cortex and hippocampus were used. The ceAPPswePS1ΔE9/TTR−/− mice lack the gene encoding transthyretin (TTR), a molecule that is enriched in CSF and known to associate with Aβ₄₀ in both in vitro and in vivo settings. The inhibitory potency of CSF for each mouse was tested by generating plugs containing buffer, ThT, Aβ₄₀ and a given concentration of CSF. Titrations were performed by changing the relative flow rates of all inlet streams to generate plugs containing constant concentrations of ions and buffer, ThT and Aβ₄₀, but different concentrations of CSF. For each of the 15 different concentrations of CSF, 50 plugs were generated, giving 750 experiments for each sample of CSF. Aβ₄₀ aggregation was then monitored by the increase of ThT fluorescence. In contrast to the experiments with low ionic strength, the ionic strength of the buffer solution in CSF experiments was adjusted to accelerate aggregation and to resemble the ion composition of mouse CSF, which includes a high concentration of Ca²⁺ and Mg²⁺ ions. In accord with previous reports, the higher ionic strength led to a decrease in the lag time of aggregation of Aβ. CSF from the wild type mouse was able to inhibit Aβ₄₀ aggregation when added at concentrations higher than 1:25 dilution. On the other hand, CSF from the ceAPPswePS1ΔE9/TTR−/− mice did not display significant inhibitory effect on Aβ₄₀ aggregation at concentrations as high as 1:5 dilutions, suggesting that TTR plays a role in inhibiting Aβ₄₀ aggregation. These results are consistent with the difference in the inhibitory potency of human CSF from patients with and without Alzheimer's disease. Thus, the plug-based system has the capability to analyze volume-limited samples from mice. Plug-based microfluidics and control of surface chemistry can be used to miniaturize peptide aggregation experiments to nanoliter volumes. As will be apparent to one skilled in the art, plug-based microfluidics are compatible with other analytical methods potentially applicable to analysis of aggregation, including, but not limited to, mass spectrometry and fluorescence correlation spectroscopy. Certain embodiments of the present invention are useful for studying, for example, in vitro aggregation biophysics, for example time-controlled aging and nucleation-growth experiments of amyloid peptides. Certain embodiments of the present invention are useful for diagnostics or for monitoring potential treatments, for example through repeated analysis of CSF or brain interstitial fluid from the same live animal (including humans), for example using a chemistrode.

It will be apparent to one skilled in the art that the present invention can be used with any peptides or protein that are unstable in structure, especially amphiphilic peptides or proteins that are susceptible to adsorption to interfaces.

One embodiment of the present invention is a method of synthesizing a fluorinated surfactant of the structure CF₃(CF₂)ₙ(CH₂)ₘO(CH₂CH₂O)ₚH, wherein n=1 to 20, m=1 to 4 and p=3 to 6, comprising coupling CF₃(CF₂)ₙ(CH₂)ₘOH and HO(CH₂CH₂O)ₚH catalyzed by a trialkyl phosphine and a reagent of the form R(CO)NN(CO)R, for example where R is piperidine. It will be apparent to one skilled in the art that other reagents, including other reagents used to carry out Mitsunobo reactions, can be used for the coupling.

Certain embodiments of the present invention can be used to generate functionalizable, mobile interfaces in plug-based microfluidics. Control of interfaces is advancing studies of biological interfaces, heterogeneous reactions and nanotechnology. Self-assembled monolayers (SAMs) have been useful for such studies, however SAMs are not laterally mobile, and therefore less applicable to systems where motion along the interface is important, such as in protein crystallization or when multiple membrane-associated proteins must assemble to perform their function. Lipid-based methods such as monolayers, vesicles, black lipid membranes and supported lipid bilayers (SLBs) are typically mobile and are widely used, but they are typically less robust and stable than SAMs and increasing the throughput capacity of such experiments remains a work in progress. Lipid-based methods effectively form 2-D crystals of proteins, but successes with nucleation of 3-D crystals are rare, presumably because salts, PEG, and detergents typically used in crystallization experiments perturb lipid structures. Furthermore, using these methods it is difficult to set up the hundreds of experiments necessary for crystallization screening.

Plug-based microfluidic systems provide the ability to generate thousands of unique reaction mixtures as droplets surrounded by fluorocarbon, allowing for rapid and expansive exploration of chemical space. In certain embodiments, His-tag binding chemistry can be implemented in an RfNTA molecule, which causes specific adsorption of a protein at an interface, offering interfacial functionality and mobility. Certain embodiments of this invention can be used for the crystallization of a His-tagged reaction center, performing multiple (for example, tens, hundreds, thousands, tens of thousands, or hundreds of thousands) crystallization trials, thereby increasing the range of crystal producing conditions, the success rate at a given condition, the rate of nucleation, and/or the quality of the crystal formed.

A His-tagged green fluorescent protein (hGFP) can be used to demonstrate that RfNTA with Ni²⁺ (RfNTA:Ni) introduces specific interactions with His-tagged proteins at the plug interface. Alone, hGFP is uniformly distributed in the plug; with RfNTA:Ni, hGFP is concentrated at the plug-carrier fluid interface. This specific interaction takes place in the presence of other surfactants. Addition of n-C₈F₁₇CH₂-OEG₃ does not inhibit hGFP interfacial adsorption. Hydrocarbon detergents that solubilize membrane protein do not compete with RfNTA for the aqueous-fluorous interface and do not interfere with the surface specific interaction. All experiments contained 0.05% w/v lauryldimethylamine N-oxide (LDAO).

Control experiments showed that interfacial adsorption depends on the formation of an RfNTA:Ni:hGFP complex. When adding (a) only Ni²⁺, (b) only RfNTA, or (c) a complex of Ni²⁺ and NTA, with no fluorous tail, to hGFP, the fluorescence was uniform across the plug. Addition of EDTA (12 mM) or imidazole (120 mM) disrupted interfacial adsorption, consistent with EDTA binding tightly to Ni²⁺, preventing formation of the RfNTA:Ni:hGFP complex and excess imidazole outcompeting His-tagged proteins for interaction with RfNTA:Ni.

The functionalized interface remains mobile, as shown by fluorescence recovery after photobleaching (FRAP) experiments. The rate of recovery is consistent with a diffusion coefficient D₀=1.1-3.1×10⁻¹² m²/sec, lower than for simple diffusion (D₀=9×10⁻¹¹ m²/sec), but higher than for diffusion in lipid monolayers or bilayers (D₀=1.3×10⁻¹³ m²/sec), presumably because the less densely packed interface was more fluid. Decreasing the packing density increased the rate of recovery.

RfNTA:Ni can be used under harsh conditions such as those encountered during protein crystallization because, like other fluorinated molecules containing NTA, such as those disclosed in U.S. Ser. No. 10/576,767, incorporated herein by reference in its entirety, it resists interference by detergents. RfNTA:Ni can aid crystallization in at least the following ways: (1) by increasing the range of conditions for crystallization and the success rate at a given condition, for example by lowering the nucleation barrier; (2) by increasing the rate of protein crystallization, for example by forming nuclei more quickly; (3) by improving crystal quality, for example by increasing order via the orienting effect at the interface.

The His-tagged reaction center (hRC) from Rhodobacter sphaeroides has been examined. Using previously known techniques, crystals irreproducibly form over 5 days at a high precipitant concentration. With only Ni²⁺ added, all nucleation is suppressed, indicating that Ni²⁺ inhibits crystal formation. With only RfNTA added, the number of plugs with crystals and the quality of crystals did not substantially change, but crystals formed more rapidly, possibly due to a residual amount of divalent cation present in the solution. When both RfNTA and Ni²⁺ were added, the number of nucleation events increased significantly and crystals formed within 1 day. In addition, crystallization occurred at lower concentrations of precipitant, and the quality of diffraction increased. For high concentrations of RfNTA:Ni at the highest concentration of precipitant, formation of crystals actually decreased. Without wishing to be bound by theory, this may be due to either interference from nonspecific adsorption through surface histidines or to nucleation occurring too rapidly to allow for ordered growth. When 10 mM imidazole was added the rate of crystal formation, the number of crystals, and the quality of crystals were improved.

The present invention can be used for the rapid generation of mobile, functionalized interfaces for exploring large areas of chemical space, both in solution and at the interface. Surface composition can be varied by changing the tagging functionalities of surfactants at the interface, or by varying the tagged molecules in solution. Such variations allow application of this method to other processes, such as nucleation of amyloid protein aggregates, multicomponent interfacial assembly, and capture assays that rely on binding to the interface and do not require washing steps to detect binding. Additional analytical techniques compatible with plugs, such as in-situ x-ray diffraction, mass-spectrometry and fluorescence correlation spectroscopy expand the applicability of the method.

### Methods

### Fabrication of a Microfluidic Device for Aggregation Experiments.

Microchannels were fabricated as disclosed in U.S. Pat. No. 6,767,194, U.S. Pat. No. 7,323,143 and U.S. Pat. No. 6,645,432, herein incorporated by reference in their entirety. Briefly, microchannels with rectangular cross section were obtained by rapid prototyping in poly(dimethylsiloxane) (PDMS). The PDMS/PDMS devices were sealed using a Plasma Prep II plasma cleaner. After sealing, the microchannels were rendered hydrophobic by baking the PDMS devices in a 120° C. oven for over one hour.

### ThT Aggregation Assay.

Aβ₄₀ (NH₂-DAEFR⁵HDSGY¹⁰EVHHQ¹⁵KLVFF²⁰AEDVG²⁵SNKGA³⁰IIGLM³⁵VGGVV⁴⁰—COOH) was obtained from rPeptide. HiLyte and fluorescein labeled Aβ₄₀ were obtained from Anaspec. To dissolve preformed peptide aggregates of Aβ₄₀, the lyophilized peptide was first dissolved in hexafluoroisopropanol (HFIP). The HFIP was evaporated from the peptides samples and the samples were then dried under high vacuum overnight. The concentration of the peptide solution was determined by absorbance at 280 nm using an extinction coefficient of ε=1360 M⁻¹ cm⁻¹ for Tyr. Aβ₄₀ was dissolved in water shortly before use. Aggregation of Aβ₄₀ was detected by Thioflavin T (ThT) fluorescence. Aggregation experiments in a well plate were done on a Polarstar Omega (BMG Labtech). For this, 50 μM Aβ₄₀ and 50 μM ThT dissolved in 50 mM Tris (pH 7.3) at 37° C. was used.

### Microfluidic Experiment.

Aqueous and fluorous solutions were filled into 10 μL gastight syringes (Hamilton Company), which were then connected to the microfluidic device by 30-gauge Teflon tubing (Weico-Wire & Cable). For Aβ₄₀ and CSF solutions, syringes were filled with water and aspirated 2 μL of either solution into Teflon tubing, which were than connected to a syringe and PDSM device. Syringes were driven by PHD 2000 Infusion pumps (Harvard Apparatus). Fluorinert FC-40 or FC-70 (3M), purified by distillation before use, was used as a carrier fluid. The fluoro-surfactant CF₃(CF₂)₇CH₂O(CH₂CH₂O)₃H (n-C₈F₁₇CH₂-OEG₃) was synthesized as described herein.

### Determination of the Concentration of Aβ₄₀ in Plugs.

Various concentrations of fluorescein-labeled Aβ₄₀ were encapsulated in plugs. Additionally, a standard curve with fluorescein (dissolved in 0.05% NH₄OH) encapsulated in plugs was recorded. A linear behavior of the fluorescence intensity of Aβ₄₀ in plugs with increasing Aβ₄₀ concentration was observed (R² value 0.99). Furthermore, the fluorescence intensities of the peptide in the plugs were in agreement with the standard curve, indicating that Aβ₄₀ was not lost during the encapsulation process. The absolute fluorescence intensity was obtained using a Lecia DMI 6000B epi-fluorescence microscope with a 10×0.4 objective coupled to a Hamamatsu ORCA ERG 1394 CCD camera with an exposure time of 3 ms. This procedure was used for both the fluorescein standard curve and the labeled Aβ₄₀.

### In Vitro Aggregation Experiments in Plugs.

Experiments were performed with a four-channel inlet device. Aqueous Aβ₄₀, Tris buffer, and a solution of ThT were loaded into syringes, and syringes were connected to the convening channels with cross sectional diameters of 225×200 μm² of a microfluidic device. The final concentrations of Aβ₄₀, ThT, and Tris (pH 7.3 at 37° C.) in plugs were always 50 μM, 50 μM and 50 mM, respectively. A syringe containing water-immiscible fluorocarbon FC-70 without or with 0.75 mg/ml n-C₈F₁₇CH₂-OEG₃ was connected to a perpendicular channel. The flow of the aqueous solutions and fluorocarbon was established by driving syringes with syringe pumps. Typical flow rates for the total aqueous and fluorocarbon phase were 0.8 and 1.2 μL/min respectively. Plugs were collected in Teflon tubing with an inner diameter of 200 μm. After the last plug was formed, the syringes were disconnected and the flow was stopped. To avoid evaporation of water from the plugs during the experiments, the plugs within the Teflon tubing were inserted into oil filled glass capillaries and the capillaries were sealed with sealing wax. The capillaries were incubated at constant temperature (37° C.) and the ThT fluorescence of the plugs was measured periodically.

### Cerebrospinal Fluid Experiments.

CSF was isolated from the cisterna magna compartment as previously described in DeMattos, et al., J. Neurochem. 2002, 81, 229. In brief, mice were anesthetized with a mixture of ketamine and xylene. An incision was made from the top of the skull to the dorsal thorax, and the musculature was removed from the base of the skull to the first vertebrae to expose the meninges overlying the cisterna magna. The tissue above the cisterna magna was excised. A microneedle was used to punctuate the arachnoid membrane covering the cistern. The CSF, which is under positive pressure as a result of blood pressure, respiration and positioning of the animal, began to flow out the needle entry site once the needle was removed, and was collected using a polypropylene narrow bore pipette as it exited the compartment. Experiments with mouse CSF were done using a five-channel inlet device, where (1) Aβ₄₀, (2) buffer, (3) ThT in a salt solution, (4) CSF, and (5) FC-40 with 1.25 mg/ml n-C₈F₁₇CH₂-OEG₃, were each allocated to one channel. In order to screen the inhibitory potency of CSF on Aβ₄₀, three experiments were performed for each mouse CSF sample with three different pre-dilutions factors of CSF (1:10, 1:5 and 1:1). Dilutions of CSF were done with a buffer containing 150 mM NaCl and 10 mM PO₄²⁻ at pH 7.3 and 37° C. Further dilutions (1:20, 1:10, 1:5, 1:4, and 1:2.5) of the pre-diluted samples were obtained by changing the flow rates of the aqueous streams. The flow rate ratio of the total aqueous and FC-40 phase was kept constant (2 μL min⁻¹/2 μL min⁻¹). The flow rates of the Aβ₄₀ and ThT/salt solutions were also kept constant. Only the buffer and CSF streams were changed to counteract each other. The final concentration of both Aβ₄₀ and ThT in the plugs was 50 μM. The salt concentration added to the ThT solution was adjusted to obtain a salt composition of 150 mM NaCl, 3 mM KCl, 0.8 mM MgCl₂, 1.4 mM CaCl₂, and 10 mM NaH₂PO₄/Na₂HPO₄ pH 7.3 at 37° C., in a plug. Plugs were stored in Teflon tubing at 37° C. and fluorescence images of the plugs were taken periodically. For each CSF concentration series, 50 plugs were formed and of those 15 were monitored. The fluorescence intensity of the 15 plugs were monitored. The aggregation kinetics of Aβ₄₀ in dilutions of the above described buffer (1:1, 1:2, and 1:4) were monitored, showing that increasing the ionic strength of the buffer is responsible for nucleating Aβ₄₀ in plugs without a lag time.

### Data Evaluation.

The fluorescence intensity in plugs was measured by taking fluorescence micrographs of single plugs using a Leica DMI 6000 microscope with a cooled CCD camera ORCA ERG 1394 (12-bit 1344×1024 resolution) (Hamamatsu Photonics). A MetaMorph Imaging System version 6.1r3 (Universal Imaging Corp) was used for imaging acquisition and Matlab 7a (Mathworks) for image analysis. The fluorescence intensity was extracted from the images by calculating the average pixel intensity of the plugs at each time point. For comparison of the experiments in plugs and well plate the ThT fluorescence intensity was normalized to the maximal intensity at the end point of the measurement, I−I₀/Iₘₐₓ, where I, I₀ and Iₘₐₓ are the fluorescence intensity, fluorescence intensity at time point 0, and the maximal fluorescence intensity at the end point of the measurement, respectively. In CSF experiments the ThT fluorescence signal was normalized to the starting value, I−I₀, were I₀ is the average fluorescence signal determined from 15 plugs.

n-C₈F₁₇CH₂-OEG₃ Concentration Assay.

To avoid Aβ₄₀ adsorption to the plug interface, it is preferable to use the correct concentration of n-C₈F₁₇CH₂-OEG₃ in the carrier fluid. It was found that a concentration lower than 0.5 mg/ml in FC-70 is less preferred due to Aβ₄₀ adsorption to the plug interface. For the carrier fluid FC-40 Aβ₄₀ adsorption can be avoided by using concentrations of n-C₈F₁₇CH₂-OEG₃ above 0.75 mg/ml. Therefore saturation of the plug surface with surfactant molecules is dependent on the carrier fluid. n-C₈F₁₇CH₂-OEG₃ concentrations above 1 and 1.5 mg/ml, however, lead to aqueous micelle formation and plug coalescence in FC-70 and FC-40, respectively. ThT fluorescence in plugs formed in the stream of FC-70 with aggregated Aβ₄₀ after 250 hours is distributed over the whole plug volume, whereas without n-C₈F₁₇CH₂-OEG₃ or at low concentrations of n-C₈F₁₇CH₂-OEG₃ the fluorescence signal is located at the plug interface. Without wishing to be bound by theory, this finding supports that aggregation of Aβ₄₀ is caused by adsorption to unfavorable interfaces.

### Chemicals and Materials for RfNTA Experiments.

All solvents and salts used in buffers purchased from commercial sources were used as received unless otherwise stated. Tris(hydroxymethyl)aminomethane (Tris base) was obtained from Fisher Scientific. Poly(dimethylsiloxane) (PDMS, Sylgard 184 Silicone Elastomer kit) was obtained from Dow Corning. FC-40 (a mixture of perfluoro-tri-n-butylamine and perfluoro-di-n-butylmethylamine) was obtained from 3M. Teflon capillaries (OD 250 μm, ID 200 μm and OD 150 μM, ID 50 μM) were received from Zeus Inc. Thirty-gauge Teflon tubing was obtained from Weico Wire & Cable. Gastight syringes were obtained from Hamilton Company.

n-C₈F₁₇CH₂-OEG₃ Synthesis.

Triethylene glycol (Acros, MW: 150.17) was dried over molecular sieves and 1 equivalent was added to a dried round bottom flask with stir bar. 1.5 equivalents of CF₃(CF₂)₇CH₂OH (Sigma-Aldrich, MW: 450.1) and 1.3 equivalents of 1,1′-azodicarbonyldipiperidine (ADDP) (TCI, MW: 252.31) were also added to the flask. These were dissolved in anhydrous benzene (Sigma-Aldrich), so that concentrations are around 0.1 M. The reaction mixture was then stirred and heated to about 60° C. Then 1.4 equivalents of tributylphosphine (Sigma-Aldrich, MW: 202.32) were added to benzene (about half the volume that was added to the round bottom flask) in an addition funnel. This solution was then slowly added to the reaction mixture over 1-2 hours. Once addition was complete the mixture was heated for an additional 3 hours, or until color was lost. Upon cooling, a significant amount of precipitant formed. The precipitant was filtered off and solvent removed by rotary-evaporation. The crude product was then rinsed and filtered with cold ethyl acetate, the solvent removed by rotary-evaporation and the process repeated in cold methanol to remove all solid impurities. The product was then purified by column chromatography and fluorous chromatography (Fluorous Technologies). Spectral data: ¹H NMR (CDCl₃) δ: 3.98 (t, 2H), 3.74 (t, 2H), 3.67 (t, 2H), 3.62 (m, 6H), 3.55 (t, 2H), 3.0 (b, 1H); ¹³C NMR (CDCl₃) δ: 120-105, 72.65, 72.44, 70.86, 70.64, 70.43, 68.35 (t, J=100 Hz), 61.73. Electrospray mass spectrometry in positive mode showed a single peak at 583.0 m/z corresponding to [n-C₈F₁₇CH₂-OEG₆+H⁺]⁺.

### Synthesis of n-C₈F₁₇CH₂-OEG₆

Hexaethylene glycol (Sigma-Aldrich, MW: 282.33) was dried over molecular sieves and 1 equivalent was added to a dried round bottom flask with stir bar. 2.5 equivalents of CF₃(CF₂)₇CH₂OH (Sigma-Aldrich, MW: 450.1) and 1.5 equivalents of 1,1′-azodicarbonyldipiperidine (ADDP) (TCI, MW: 252.31) were also added to the flask. These were dissolved in anhydrous benzene (so that concentrations were around 0.1 M). The reaction mixture was then stirred at room temperature. Then 1.6 equivalents of tributylphosphine (Sigma-Aldrich, MW: 202.32) was dissolved in benzene (about half the volume that was added to the round bottom flask) in an addition funnel. This solution was then slowly added to the reaction mixture over 3-4 hours, and the reaction allowed to run overnight. The precipitant was filtered off and solvent removed by rotary-evaporation. The crude product was then rinsed and filtered with cold ethyl acetate, the solvent removed by rotary-evaporation and the process repeated in cold methanol to remove all solid impurities. The product was then further purified by column chromatography and fluorous chromatography. Spectral data: ¹H NMR (CDCl₃) δ: 4.02 (t, 2H), 3.74 (t, 2H), 3.68 (t, 2H), 3.61 (m, 18H), 3.56 (t, 2H), 3.0 (b, 1H); ¹³C NMR (CDCl₃) δ: 120-105, 72.65, 72.37, 70.76, 70.72, 70.67-70.59, 70.37, 68.33 (t, J=100 Hz), 61.73. Electrospray mass spectrometry in positive mode showed a single peak at 715.0 m/z corresponding to [(1)+H⁺]⁺.

### Synthesis of Compound 2 (See Scheme 1)

2 equivalents of 1,1′-carbonyldiimidazole (Sigma-Aldrich, MW: 162.15) was dissolved in dry methylene chloride (Fisher). (1) (MW: 714.41) was also dissolved in dry methylene chloride and added slowly to the 1,1′-carbonyldiimidazole. The reaction was stirred overnight at room temperature under dry nitrogen. The product Compound 2 was purified by column chromatography. Spectral data: ¹H NMR (CD₃OD) δ: 8.33 (s, 1H), 7.63 (s, 1H), 7.09 (s, 1H), 4.1 (t, 2H), 3.88, (m, 2H), 3.78 (m, 2H), 3.61 (m, 20H), 2.0 (b, 1H).

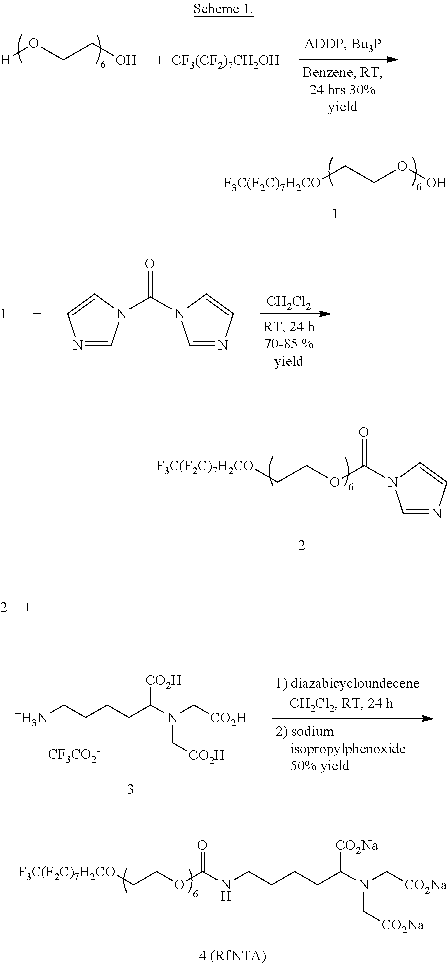

### Synthesis of RfNTA (Compound 4)

RfNTA is synthesized by attaching a nitrilotriacetate (NTA) head group to n-C₈F₁₇CH₂-OEG₆ (Compound 1, See Scheme 1). n-C₈F₁₇CH₂-OEG₆ was synthesized as described herein using Mitsunobu conditions. 10 equivalents of 1,8-Diazabicyclo[5.4.0]undec-7-ene (Acros, MW: 152.24) and 1 equivalent of Nα,Nα-Bis(carboxymethyl)-L-lysine trifluoroacetate salt (Compound 3) (Sigma-Aldrich, MW:376.3) was suspended in dry methylene chloride. Then Compound 2 (MW: 808.5) was dissolved in methylene chloride and added to the reaction mixture and the reaction was stirred overnight at room temperature under dry nitrogen. The solvent was then removed and the remaining solid washed several times with EA. The solid was then dissolved in a minimal amount of methanol (Fisher) and sodium isopropylphenoxide (prepared by mixing sodium methoxide (Acros) with a slight excess of isopropylphenol (Sigma-Aldrich)) was added. The mixture was stirred for 15 minutes and any solid filtered off. The liquid was then evaporated, the solid dissolved in a minimal amount of methanol and the product was precipitated using ether (Fisher). The product Compound 4 (MW=1068.6) was then further purified using fluorous chromatography. Spectral data: ¹H NMR (CD₃OD) δ: 4.1 (m, 4H), 3.7 (m, 5H), 3.61 (m, 22H), 3.10 (m, 2H), 2.0-1.5 (m, 6H); ¹³C NMR (CDCl₃) δ: 158.94, 129.93, 73.165, 71.49-71.28, 70.85, 69.31, 68.97 (t, J:=100 Hz), 64.85, 56.86, 41.28, 30.52, 28.43, 25, 48. Electrospray mass spectrometry in negative mode showed a predominant peak at 1000.8 m/z, and an additional peak at 1023.8 m/z consistent with [RfNTA-3Na⁺+2H⁺]⁻ salt and [RfNTA-2Na⁺H⁺]⁻ respectively. It will be apparent to one skilled in the art that RfNTA can be converted to an acid, or can be isolated as a partially or fully deprotonated compound together with singly or multiply charged cations.

### Characterization.

¹H and ¹³C NMR spectra were acquired on Bruker Avance DRX 400 MHz or Bruker Avance III 500 MHz spectrometers (Bruker BioSpin). ESI-MS were acquired from Agilent 1100 LC/MSD with ESI or APCI ion sources (Agilent Technologies).

### PDMS Device Fabrication for RfNTA Experiments.

PDMS was used to fabricate all microfluidic devices. Microchannels with rectangular cross-sections were fabricated with rapid prototyping as described elsewhere herein. The hybrid microfluidic devices consisted of PDMS microchannels (cross-section: 250 μm by 250 μm) with three or four tapered inlets (100 μm by 250 μm) for aqueous phases and one orthogonal inlet for carrier fluid to form nanoliter plugs. The channel walls were functionalized with (tridecafluoro1,1,2,2-tetrahydrooctyl)-1-trichlorosilane (United Chemical Technologies) to render them hydrophobic and fluorophilic. Teflon tubing was inserted into the outlet up to the plug forming junction. The tubing was sealed to the device with either wax or PDMS glue.

Interfacial Adsorption Experiments Using hGFP.

Three-aqueous-inlet devices were used to perform interfacial adsorption experiments. In these experiments the left aqueous stream contained the additive solution, which was either (1) 100 mM Tris, pH 7.0, (2) 115 μM NiSO₄ in 100 mM Tris, pH 7.0, (3) 125 μM RfNTA in 100 mM Tris, pH 7.0, (4) 125 μM Compound 3 and 115 μM NiSO₄ in 100 mM Tris, pH 7.0, or (5) 62.5 μM RfNTA and 57.5 μM NiSO₄ in 100 mM Tris, pH 7.0. The stock buffer solution had been pretreated with Chelex to remove any divalent cations and all other solutions were made from this. When mixed with RfNTA the concentration of Ni²⁺ was always between 90-95% of the RfNTA concentration to ensure no free Ni²⁺ was left in solution. Higher concentrations of RfNTA:Ni were also tested and performed similarly. The center stream always contained 100 mM Tris, pH 7.0 except for an experiment with 20 mM EDTA (Fisher) in 100 mM Tris, pH 7.0. The right stream contained 2 μM hGFP (Upstate, now part of Millipore) and 0.25% lauryldimethylamine N-oxide (LDAO) (Anatrace) (w/v) in 100 mM Tris, pH 7.0. Plugs were formed into Teflon tubing (OD 150 μm, ID 50 μm) using FC-40 that had been pre-equilibrated with 20 mM EDTA. The carrier fluid flowed at a rate of 8.3-16.67 nL/sec, using PHD2000 syringe pumps (Harvard Apparatus). No additional surfactant was used in the FC-40 except when 0.25 mg/mL n-C₈F₁₇CH₂-OEG₃ (MW 582.25) was used. The hGFP always flowed at 3.33 nL/sec so the final concentration was 400 nM hGFP and 0.05% LDAO. The other two flow rates were adjusted so the total aqueous flow rate was always 16.67 nL/sec. The additive and buffer flow rates were both 6.67 nL/sec for additive conditions 1-4, so the final concentrations were 46 μM Ni²⁺, 50 μM RfNTA, and 50 μM Compound 3 and 46 μM Ni²⁺, for conditions 2-4 respectively. For condition 5 the flow rates were 3.33 and 10 nL/sec for the additive and buffer stream leading to final concentrations of 12.5 μM RfNTA and 11.5 μM Ni²⁺. When EDTA was added to the buffer stream it gave a final concentration of 12 mM EDTA. Plugs were formed for at least 2 minutes, then flow was stopped, the tubing cut and ends sealed onto a glass slide with wax. For imaging the tubing was submerged in milliQ-H₂O. Bright field and fluorescence images were obtained using confocal microscopy on a Leica DMI6000 microscope (Leica Microsystems) using a VT-Infinity 3 confocal scanning head (VisiTech international). They were analyzed using SimplePCI software (Hammamatsu Corp.), and MetaMorph 6.3 (Molecular Devices).

### FRAP Experiments.

Plugs were formed as described elsewhere herein. The final concentration of His 10 was 3.7 μM in 20 mM Tris pH 8.0. The final RfNTA:Ni concentration was 30 μM RfNTA and 28.5 μM Ni. Plugs were formed in three different fluorocarbons: FC-70 with a viscosity of 12 cSt, FC-40 with a viscosity of 1.8 cSt, and FC-84 with a viscosity of 0.53 cSt at room temperature. All fluorocarbons contained 170 μM n-C₈F₁₇CH₂-OEG₃ to prevent nonspecific adsorption.

During the FRAP experiments 10 images of the edges of a plug were obtained before bleaching. Next, spots were bleached on the edges of the plugs using high laser power for 2 seconds, and the regions were then monitored for an additional 40 seconds. Linescans spanning the edges of the plugs were used to monitor fluorescence recovery at the plug interface over time. The shape of the bleach spot was fit using Origin 8 (OriginLab) to the Gaussian curve described by Equation 1. A generalized solution to Fick's second law of diffusion is given in Equation 2 and draws obvious parallels to Equation 1 (See Seiffert, S, and Oppermann, W., J. Microsc.—Oxf. 2005, 220, 20-30).

$$\begin{matrix}I ( x , t ) = {I}_{0} - A ( t ) * {e}^{\frac{- {( x - {x}_{0} )}^{2}}{2 * {w}^{2}}} & Equation 1 \\ C ( r , t ) = {C}_{0} - \frac{M}{{( 4 π Dt )}^{d / 2}} * {e}^{\frac{- {r}^{2}}{4 Dt}} & Equation 2\end{matrix}$$

I is intensity, I₀ is the initial intensity, A(t) is the maximum amplitude at time t, x is the position, and x₀ is the offset, and w determines the distribution width of the curve. C is the concentration, C₀ is the initial concentration. M is the total amount of the diffusing species, D is the diffusion coefficient, r is the position, and t is time.

Monitoring the change in the shape of the Gaussian over the time course of recovery allows for extraction of a diffusion coefficient and requires no predetermined knowledge of the geometry of the bleach spot or of the diffusion dimension (See Seiffert, S, and Oppermann, W., J. Microsc.—Oxf. 2005, 220, 20-30). From Equations 1 and 2 it can be seen that w²=2*D*t, so plotting w² vs. t should give a straight line with slope 2*D. The diffusion dimension can be determined using log A(t)=−d/2*log(t)+K.

This analysis gave diffusion coefficients of 7.3±2.6×10⁻¹² m²/sec, 15.2±2.4×10⁻¹² m²/sec, 39.2±11.5×10⁻¹² m²/sec for FC-70, FC-40 and FC-84 respectively. In all cases there was substantially complete recovery of fluorescence indicating that no immobile phase existed. The measured diffusion dimension was 1.1±0.3, 1.1±0.2, 2.0±0.4 for FC-70, FC-40 and FC-84 respectively. However, due to the log-log plot, the accuracy of d is greatly affected by the time range in which reliable data can be obtained. Divalent metals are capable of quenching fluorescein, and significant quenching was observed when using His10. This led to fairly noisy data, which limited the timescale over which accurate measurements could be obtained. Analysis that included later time points indicated that d for FC-70 was likely accurate; whereas d for FC-40 fell between 1 and 2, and d for FC-84 remains near 2. This general trend of increasing diffusion dimension with faster diffusion can be explained if the geometry of the bleaching zone is considered. The bleaching zone is not a point; rather, there is a cone above and below the focal plane. If the bleaching zone of the laser above and below the focal plane is large compared to the length scale of diffusion, then the predicted diffusion dimension would be 1, as recovery can only come from fluorophore in the focal plane. On the other hand, for faster diffusion, the diffusion dimension would approach 2 as fluorophores above and below the plane can contribute.

Similar experiments were performed with hGFP at 1 μM, and fluorescein-labeled hSA at 250 nM. The hGFP could not be analyzed in the same fashion due to a quenching effect that resulted in a temporary enhancement of fluorescence around the bleach spot during recovery. Rough estimates of Dₒ, obtained by fitting fluorescence recovery of the area surrounding the bleach spot with a single exponential curve, were approximately 3×10⁻¹² m²/sec for FC-40 and 30 μM RfNTA:Ni. For the labeled hSA interfacial binding was weak, possibly due to the close proximity of the His-tag to structural components of the protein. However, initial experiments resulted in D₀ using the Gaussian fitting model of 4.5±0.6 and 9.2±2.4×10⁻¹² m²/sec for FC-70 and FC-40, respectively. Without wishing to be bound by theory, the hGFP and hSA are thought to give slower diffusion than His10 because a protein would create more drag than a small peptide. However the D₀ for a protein in solution is around 1×10⁻¹⁰ m²/sec, so events at the interface still dominate the diffusion rather than the attached protein or peptide.

FRAP was measured using a Leica tandem scanner SP5 spectral confocal on a DMI6000 microscope with a 40×NA1.40 oil objective. Images were analyzed by using ImageJ software and the plug-in loci bio-formats and by using Mathematica.

### Tensiometry.

Droplets of fluorous surfactant solution were formed at the end of disposable droplet extrusion tips. The tips were assembled by using quick-set epoxy to glue polyimide-coated glass tubing into 1-10 μL disposable pipette tips that were oxidized in a Plasma Prep II plasma cleaner (SPI Supplies) for 3 min to render them hydrophilic. The end of the capillary was positioned just inside the end of the pipette tip. The polyimide tubing was connected to a 50 μL Hamilton Gastight syringe using 30-gauge Teflon tubing. The syringe was filled with the fluorous solution. The pipette tip was positioned so that it sat in the aqueous solution within a 1 mL polystyrene cuvette and held in place by a clamp. The formed droplets were imaged using Model 250 Standard Digital Goniometer & DROPimage Advanced software (Rame-Hart Instrument Co).

### Crystallization of Reaction Center (RC).

Four-aqueous-inlet devices were used to perform crystallization experiments. In sequence, the precipitant stream was 50% (w/v) PEG 4000, 1.1 M NaCl, 9% (w/v) 1,2,3-heptanetriol (HPT) in 50 mM Tris pH 7.8. The buffer stream was 9% HPT in 50 mM Tris pH 7.8. The additive stream used six different solutions in six different sets of experiments, and they were (1) 10 mM Tris pH 7.8, which was defined as the standard condition, (2) 45 μM NiSO₄ in 10 mM Tris pH 7.8, (3) 50 μM RfNTA in 10 mM Tris pH 7.8, (4) 50 μM RfNTA and 45 μM NiSO₄ in 10 mM Tris pH 7.8, (5) 200 μM RfNTA and 180 μM NiSO₄ in 10 mM Tris pH 7.8, and (6) 200 μM RfNTA and 180 μM NiSO₄ and 10 mM imidazole in 10 mM Tris pH 7.8. The protein stream was 6 mg/mL hRC from Rhodobacter sphaeroides in 0.05% (w/v) LDAO and 10 mM Tris pH 7.8. The fluorinated carrier fluid was FC-40. The carrier fluid, protein, and additive streams were maintained at constant flow rates of 41.7 nL/sec, 13.3 nL/sec and 3.3 nL/sec, respectively. The flow rate of the precipitant stream was first increased from 8.3 nL/sec to 15 nL/sec, and then decreased from 15 nL/sec to 8.3 nL/sec, with a step size of 1.7 nL/sec. Correspondingly, the buffer stream was first decreased from 8.3 nL/sec to 1.7 nL/sec, and then increased from 1.7 nL/sec to 8.3 nL/sec, with a step size of 1.7 nL/sec. Each flow rate step lasted for 14 s. After one step was finished, the subroutine was stopped and, ˜5 s later, it was restarted with the setup of the next step. In this experiment, two sets of plugs generated from identical flow rates were counted as duplicates. The experiments were performed six times with six different additive solutions. The trials, in the form of plugs, were transported and stored in Teflon tubing (O.D.: 250 μm and I.D.: 200 μm) which was sealed inside glass tubing (O.D.: 3 mm and I.D.: 1.8 mm) prefilled with FC-70. The experiment was performed under dim light, and the trials were kept in the dark at 23° C.

The following final precipitant concentrations were obtained: Concentration A, 22.5% (w/v) PEG 4000, 0.495 M NaCl, 4.5% (w/v) 1,2,3-heptanetriol (HPT) in 30 mM Tris pH 7.8; Concentration B, 20% (w/v) PEG 4000, 0.44 M NaCl, 4.5% (w/v) 1,2,3-heptanetriol (HPT) in 30 mM Tris pH 7.8; Concentration C, 17.5% (w/v) PEG 4000, 0.385 M NaCl, 4.5% (w/v) 1,2,3-heptanetriol (HPT) in 30 mM Tris pH 7.8; Concentration D, 15% (w/v) PEG 4000, 0.33 M NaCl, 4.5% (w/v) 1,2,3-heptanetriol (HPT) in 30 mM Tris pH 7.8; and Concentration E, 12.5% (w/v) PEG 4000, 0.275 M NaCl, 4.5% (w/v) 1,2,3-heptanetriol (HPT) in 30 mM Tris pH 7.8. Because the behavior of crystallization can be different depending on the purity of samples, the experiment was performed twice with different protein preparations made months apart, and similar results were obtained both times. To avoid spurious results from background levels of divalent metals such as those observed for condition 3, low levels of a chelating agent such as EDTA could be added to the crystallization conditions

Counting Plugs with Crystals.

Plugs were counted under dim light at day 1, day 2, day 5 and day 12 after setting up the experiments. In all experiments for each flow rate step, 40 plugs were counted starting from the tenth plug of each set. Plugs with crystals were counted as one hit. To make sure one did not start from different plugs at different days, a picture of the first counted plug in each set of plugs was taken.

### Determining the Nucleation Rate.

The nucleation rate was determined for different additive conditions at two precipitant concentrations: (1) Concentration B, where plugs of crystallization trials were formed at 13.3 nL/sec and (2) Concentration A, where plugs of crystallization trials were formed at 15 nL/sec. In (1) final concentrations of precipitant were 20% (w/v) PEG 4000, 0.44 M NaCl and 30 mM Tris pH 7.8, whereas in (2) final concentrations of precipitant were 22.5% (w/v) PEG 4000, 0.5 M NaCl and 30 mM Tris pH 7.8. To determine the nucleation rate for each concentration of precipitant, the percentage of the number of plugs with crystals out of the total number of counted plugs (40 in all cases) was plotted against the recording time (days). The nucleation rate was then extracted from the slope of the plots, determined by dividing the change in the number of plugs with crystals by the change in time. The nucleation rate was equal to the largest slope for each curve. If no crystals were obtained over time, the nucleation rate was zero. All the nucleation rates were extracted from day 0 to day 1 except the one for the standard condition at Concentration A, which was extracted from day 0 to day 5.

In these experiments, the number of plugs were counted instead of the number of crystals. For standard conditions, there was always only one crystal per plug, whereas many of the other conditions often had multiple crystals per plug. Because the number of individual nucleation events was not counted, the calculated nucleation rates represent the lower limit of the actual nucleation rate.

### Crystal Preparation and X-Ray Data Collection.

Cryo-protectant for freezing hRC crystals was 25% (v/v) PEG 400, 20% (w/v) PEG 4000, 0.44 M NaCl in 0.08% (w/v) LDAO and 20 mM Tris pH 7.8. Crystals of hRC from R. sphaeroides grown in the plugs were extracted by attaching a syringe to one end of the Teflon tubing and flowing the crystals slowly into a drop of cryo-protectant by using the manual syringe driver. Once crystals were flowed into a 2 μL cryo-protectant drop, the crystals were picked up with a CryoLoop (Hampton Research) and flash frozen in liquid nitrogen. The X-ray diffraction experiments were performed at GM/CA Cat station 23 ID-B of the Advanced Photon Source (Argonne National Laboratory). In all experiments, the wavelength was kept at 1.03 Å. A 10 μm minibeam was used with the attenuation at five. The exposure time was kept at 10 seconds. The data were processed in HKL2000 to determine the space group and diffraction limit with S/N over 2.0. Crystals from 200 μm RfNTA:Ni at Concentration B diffracted best to 3.1 Å; b: Crystals from the standard condition at Concentration A diffracted best to 4 Å and c: Crystals from 50 μM RfNTA at Concentration A diffracted best to 4 Å.

From the foregoing, it will be observed that numerous variations and modifications may be effected without departing from the spirit and scope of the invention. It is to be understood that no limitation with respect to the specific embodiment illustrated herein is intended or should be inferred. It is, of course, intended to cover by the appended claims all such modifications as fall within the scope of the claims.

What is claimed is:

1. A microfluidic system comprising:
a microchannel;
a carrier fluid comprising a fluorinated oil in the microchannel;
at least one plug comprising an aqueous plug-fluid in the microchannel and substantially surrounded on all sides by the carrier-fluid;
a fluorinated surfactant comprising a functional group capable of selectively binding a target molecule.

2. The microfluidic system of claim 1, further comprising a second fluorinated surfactant.

3. The microfluidic system of claim 2, wherein the second fluorinated surfactant comprises a functional group that does not significantly bind to the target molecule.

4. The microfluidic system of claim 1, wherein the target molecule is a biological molecule.

5. The microfluidic system of claim 1, wherein the target molecule is water soluble.

6. The microfluidic system of claim 1, wherein the functional group is selected from the group comprising a nitrilotriacetate, an iminediacetate a triazacyclononane, a biotin derivative, a glutathione derivative, a maltose derivative, an antibody, an aptamer, a thioredoxin tag, a FLAG tag, a hemaglutinin tag, and an OmpA signal sequence tag.

7. A compound of the formula 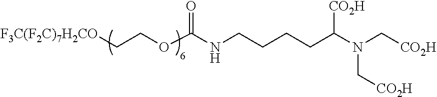
and salts thereof.

8. A method of synthesizing a fluorinated surfactant of the formula
CF₃(CF₂)ₙ(CH₂)ₘO(CH₂CH₂O)ₚH;
wherein n is an integer from 1 to 20;
m is an integer from 1 to 4;
p is an integer from 3 to 6;
comprising coupling a compound of the formula CF₃(CF₂)ₙ(CH₂)ₘOH with a compound of the formula HO(CH₂CH₂O)ₚH.
